(19) United States
(12) Patent Application Publication
(10) Pub. No.: US 2023/0201408 A1
(43) Pub. Date: Jun. 29, 2023

(54) Electronic Breathing Mask with UVC Air Purification System and Peripherals

(71) Applicant: Cedarz Group, LLC, Sachse, TX (US)

(72) Inventor: Roland Moubarak, Sachse, TX (US)

(73) Assignee: Cedarz Group, LLP, Sachse, TX (US)

(21) Appl. No.: 17/996,453

(22) PCT Filed: Nov. 4, 2020
(86) PCT No.: PCT/US2020/058900

§ 371 Date: Oct. 18, 2022

Related U.S. Application Data
(60) Provisional application No. 62/704,147, filed on Apr. 23, 2020.

(51) Int. Cl. A61L 9/20; A62B 23/02

(52) U.S. Cl. CPC A61L 9/20; A62B 23/02; A61L 2209/14; A61L 2209/111; A61L 2209/12

(57) ABSTRACT

An air purification system is described herein; it comprises a breathing mask configured to hold an ultraviolet light source trapped between two air filter membranes. The light from the ultraviolet light source shines on and sterilizes the surfaces of the membranes and the air as it passes between the filters. The mask can be configured to operate as an air purifier or can be made intelligent enough to be environment aware to alarm the user and adjust the filtering loop to accommodate the variables in the air. In addition, the mask can be configured to connect to a smart device via a wireless interface so it can act as a smart peripheral to a smart phone; the mask can also include optional industry specific sensors and peripherals. This device is envisioned to be an apparatus to the mouth which provides personal protection from the environment.

### CROSS-REFERENCE TO RELATED APPLICATIONS

This application claims priority to U.S. Provisional Patent Application No. 62/704,147 filed Apr. 23, 2020.

### BACKGROUND

This invention relates to air purification and more particularly to an aperture for subjecting air to ultraviolet (UV) light. The apparatus may be used for air sterilization and is intended as a facial mask application. The invention relates generally to devices and methods for disinfecting air and more particularly to a portable and wearable system for disinfecting air using ultraviolet light.

Wearing a mask is becoming a necessity nowadays; however, many limitations are associated with conventional masks. Conventional masks do not inactivate organic pathogens, like viruses and bacteria. Thus, pathogens trapped by conventional masks can be transferred once the masks is taken off and handled. Additionally, any pathogen that might pass through a filter of a conventional mask may harm the user of the mask if the pathogen comes from the environment. If the pathogen comes from the user of the mask, individuals who come in contact with the user of the mask may be harmed. The development of a mask that can minimize the air resistance of breathable air while providing the sterilization required to inactivate the pathogens is critical to control the spread of pathogens.

Additionally, as masks become a necessity, an opportunity exists to improve contact tracing. Conventional contact tracing relies on test results, which are often not known until days after an individual infected with a communicable disease has continued to go about their normal daily routine, spreading the communicable diseases in the process. Further, individuals infected with a communicable disease may never get tested because they do not experience symptoms. The inability to determine whether an individual is carrying a communicable disease in real time limits the ability to stop an individual from accidently spreading a communicable disease. The development of a tool that can identify signs of a communicable disease in real time is critical to control the spread of pathogens.

Further, as masks become a necessity, an opportunity exists to improve the functionality of various tools designed to project or collect audio and visual stimuli from a first person perspective. Conventional tools configured to project or collect audio and visual stimuli from a first person perspective are configured to be mounted to a torso of an individual or a headband of an individual. The existing mounting designs fail to accurately track where the user is actually looking. For example, a camera mounted to a user's torso tracks where the torso faces, not where the user's eyes face. Thus, improper video streams can accidently be obtained. The development of mask accessories that incorporate tools configured to operate from a first person perspective will improve the functionality of the first person tools.

### SUMMARY

It is an aspect of the present invention that a wearable air purifier employing UV radiation is able to inactivate all viruses and bacteria from breathable air thus serving as a personal protective device. The configuration of the UV radiation in conjunction with an air filter of the present invention enables deep penetration of the UV radiation into the air filter.

It is another aspect of the present invention that an electronic module can be configured to operate in conjunction with the wearable air purifier. The electronic module can include various tools. The tools may be configured to collect data such that real time identification of surrounding environmental hazards may be identified. The tools may also be configured to collect data corresponding to a user's health in real time. The electronic module may be configured to communicate with a smart device or a network for purposes including but not limited to contact tracing. Additionally, the electronic module may be configured to include tools designed to function from a first person perspective such that the functionality of the tools improves as a result of more accurate first person tracking.

This protective device with a battery and communication interface can also serve to provide industry specific features to the person wearing it. Hence, this device can be equipped with sensors that can be customized to a certain industry including mining, medical, police, first responders and military applications.

The featured applications of the present invention, illustrated in FIGS. 1-9, are exemplary and are not intended to define the scale, exact shape, function, and/or location of each device or interface.

### BRIEF DESCRIPTION OF DRAWINGS

The present application includes the following figures that are provided as illustrative embodiments of the inventions contained in the present disclosure:

FIG. 1 is a side view of an example of an air purification system.

FIG. 2 is a top view of an example of an air purification system including a mechanism for securing a filter.

FIG. 3A is a front view of an aperture of an air purification system configured to receive a filter frame.

FIG. 3B is a rear view of a filter frame of an air purification system with at least one projection.

FIG. 4 is a side view of a frame of an air purification system with at least one projection.

FIG. 5A is a front view of an aperture of an air purification system configured to receive a deformable filter frame

FIG. 5B is a rear view of a deformable filter frame of an air purification system.

FIG. 6 is a top view of an example of an air purification system showing UV radiation beams.

FIG. 7 is a side view of an example of an electronic module for an air purification system.

FIG. 8A is a prospective view showing, as separate components, an electronic module for an air purification system and a frame of an air purification mask configured to receive the electronic module.

FIG. 8B is a prospective view showing, coupled together, an electronic module for an air purification system and a frame of an air purification mask configured to receive the electronic module.

FIG. 9 is a front view of an electronic module for an air purification system.

FIG. 10 is a side view of an air purification system showing a projection from the air purification system.

### DETAILED DESCRIPTION

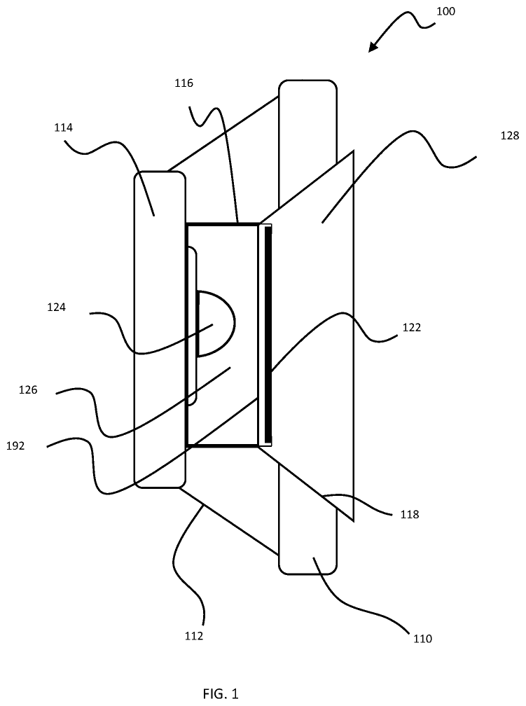

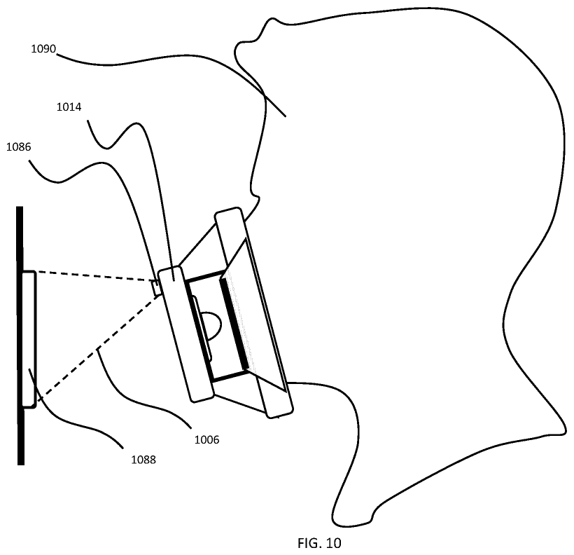

FIG. 1 shows in an illustrative embodiment according to the present disclosure where an air purification system 100 may include a gasket 118, a first filter 122, a second filter 116, and an ultraviolet (“UV”) light source 124. In some implementations, the gasket 118 and the first filter 122 define at least a portion of a first chamber 128. The gasket 118 may be configured to interface with a face of a user (e.g., 1090 in FIG. 10) as illustrated by the embodiment shown in FIG. 10 and the first chamber 128 may be further defined in part by the face of the user. The first filter 122 and the second filter 116 may be configured to define at least a portion of a second chamber 126, and the UV light source 124 may be disposed within the second chamber 126. The second filter 116, the second chamber 126, the first filter 122, and first chamber 128 may be configured to direct airflow from atmosphere to a respiratory system of the user during inhalation. The system may be configured to purify air, and the purified air may include the air that flows from the atmosphere to the respiratory system of the user. In some embodiments, placement of the UV light source 124 in the second chamber 126 that air passes through before being inhaled by a user enables the system to expose the air and the particles in the air to UV-C radiation prior to being inhaled by the user. By exposing the air to the UV-C radiation, unwanted particles contained in the air can be eliminated, modified, disinfected, inactivated, killed, or a combination thereof. The unwanted particles may include different pathogens. As non-limiting examples, the different pathogens may include viruses, bacteria, fungi, spores, environmental contaminants, or a combination thereof. The UV-C radiation may be configured to kill or inactivate the different pathogens. A person of ordinary skill will appreciate that UV light in the second chamber 126 irreparably damages the DNA and/or RNA of the different pathogens, rendering them incapable of infecting a host. Some embodiments may improve inactivation of the different pathogens by increasing exposure time to UV-C light. For example, the first filter 122 may impede the movement of a pathogen through the second chamber 126, thereby increasing exposure time to the UV-C radiation. Some embodiments may improve inactivation of the different pathogens by exposing the pathogen to varying wavelengths of UV-C radiation. The wavelength that the pathogen is exposed to may be based on the type of pathogen a user is exposed to.

The gasket 118 may be made of, for example, rubber, silicon, polymers, or a combination thereof. In embodiments where the gasket 118 is made of a soft material such as rubber, the gasket 118 may be comfortable for the user. In some embodiments, the gasket 118 may surround a mouth and a nose of the user (e.g., 1090 in FIG. 10). The gasket 118 may be configured to form an air tight seal with the face of the user. In embodiments where the gasket 118 surrounds the mouth and nose of the user and forms an air tight seal, the system is able to create an air flow within the system based on a force generated when the user inhales and exhales. In some embodiments, the air is configured to enter and/or exit the second chamber 126 from below the mask such that if a user (e.g., 1090 in FIG. 10) wears glasses, the glasses will not fog. In some embodiments, the gasket 118 may be coupled to the first filter 122. The gasket 118 may be configured to be coupled to a first structural component 110. The first structural component 110 may be coupled to an electronic module 114 positioned opposite the gasket 118 via a second structural component 112. The first structural component 110 and the second structural component 112 may define a frame that support, defines, and/or holds the gasket 118, the second filter 116, the first filter 122, the first chamber 128, and/or the second chamber 126. The first and second structural components may, for example, be comprised of plastic, composite, metals, or a combination thereof. Further these first and second structural components may comprise multiple subcomponents to provide the structure, frame, or support described by example herein.

The first filter 122 may be disposed between the first chamber 128 and the second chamber 126. Airflow from the atmosphere to the respiratory system of the user may be configured to pass through the first filter 122. In some embodiments, the second filter 116 may be disposed between the second chamber 126 and the atmosphere. The airflow from the atmosphere to the second chamber 126 may be configured to pass through the second filter 116.

The first filter 122 may be configured to prevent a first size particle from passing through the first filter 122. The first size particle may be less than or equal to 0.1 microns. In other implementations, the first size particle may be less than, equal to, or between any two of the following: 0.01, 0.02. 0.03, 0.04, 0.05, 0.06, 0.07, 0.08, or 0.09 microns. The first filter 122 may include a plurality of holes configured to prevent the first size particle from passing through the first filter 122. In some embodiments, the first filter 122 may include a plurality of layers of fabric including the plurality of holes configured to prevent the first size particle from passing through. The plurality of layers of fabric may be randomly arranged such that the plurality of holes do not align. Particles that can pass through the holes are thus impeded from passing through the first filter 122 such that exposure time of particles to the UV-C radiation is increased. The size of the first particle may be selected based on a size of unwanted particles contained in the air to protect the user from inhaling the unwanted particles. Particles may include, for example, pathogens, dust, chemical, or other undesired components from a user's atmosphere.

In some embodiments, the first filter 122 is a membrane. The first filter 122 may be made from natural or synthetic fibers, plastics, or cloths. The second filter 116 may be the same material as the first filter 122 or may be a different material from the first filter 122. The second filter 116 may also be a membrane. In some embodiments, the second filter 116 may be washable. When the second filter 116 is washable, the user is able to wash out the large particles stopped by the second filter 116 rather than having to replace the second filter 116. In some embodiments, the second filter 116 may be replaceable.

The second filter 116 may be configured to prevent a second size particle from passing through the second filter 116. The second size particle may be greater than the first size particle. The UV light source 124 may be placed in the second chamber 126. By preventing larger particles from entering the second chamber 126, the overall number of particles within the second chamber 126 may be reduced such that the particles within the second chamber 126 are more likely to receive UV-C radiation from the UV light source 124. In some embodiments, the second size particle may be less than 5 microns. In other implementations, the second size particle may be less than, equal to, or between any two of the following: 0.1, 5, 10, 20, 50, 100, 150, 200, or 250 microns. The size of the particle blocked by second filter 116 may be selected such that the second filter 116 blocks items that are large enough to block UV-C radiation from the UV light source 124 from interacting with a pathogen in the second chamber 126. For example, an item large enough to block the UV-C radiation may include sand, dust, or a combination thereof. The second filter 116 may include a plurality of holes where the plurality of holes are configured to prevent the second size particle from passing through the second filter 116. In other embodiments, the second filter 116 may include a plurality of layers of a second fabric including the plurality of holes configured to prevent the second size particle from passing through. The plurality of layers of the second fabric may be randomly arranged such that the plurality of holes do not align.

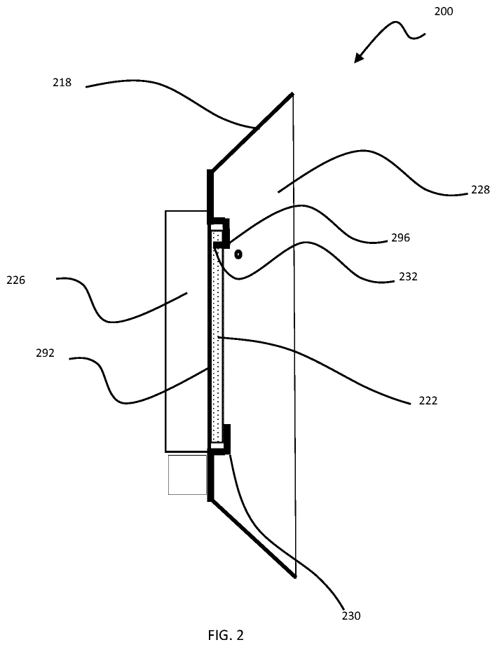

FIG. 2 shows some embodiments where, for example, an internal filter 222 (e.g., 122 in FIG. 1) is configured to be removable from the system 200. In some embodiments, a first chamber 228 may be defined at least in part by a mask frame 230 configured to support the internal filter 222. The mask frame 230 may define an opening configured to support the internal filter 222 such that the internal filter 222 cannot move along at least two axes. The system may include a latch 232 configured to rotate about a hinge 296 to secure the internal filter 222 within the mask frame 230. By rotating the latch 232 about the hinge 296, the internal filter 222 may be removed from the mask frame 230. The mask frame 230 may be interposed between the first chamber 228 and the second chamber 226. In some embodiments, the mask frame 230 may be positioned on an opposite side of the first chamber 228 than the face of the user (e.g., 1090 in FIG. 10). By enabling the internal filter 222 to be removable, a user can replace the internal filter 222 after a certain amount of time has passed or a certain number of particles have been trapped within the internal filter 222. In some embodiments, mask frame 230 also comprises a gasket 218 that may create an air tight seal with a user's face. While FIG. 2 shows the mask frame 230 positioned in the first chamber 228, in some embodiments, the mask frame 230 may be positioned within the second chamber 226.

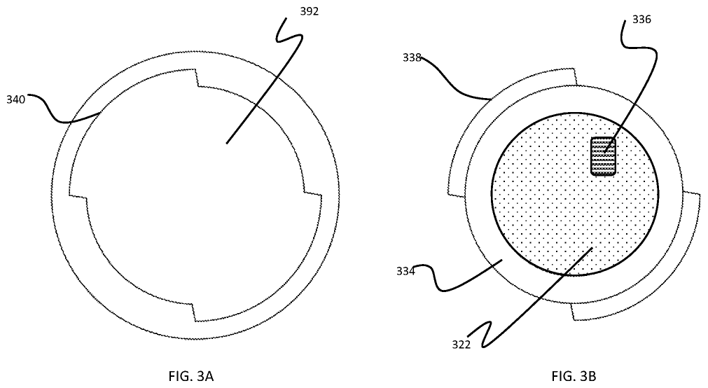

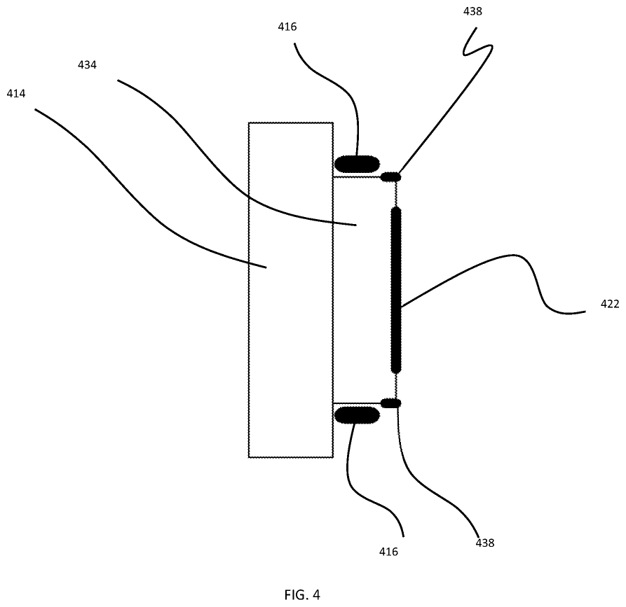

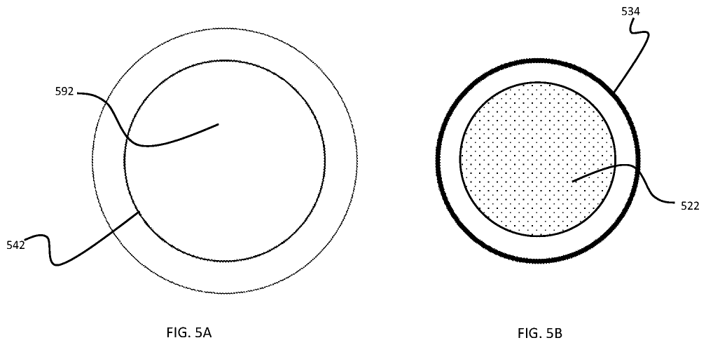

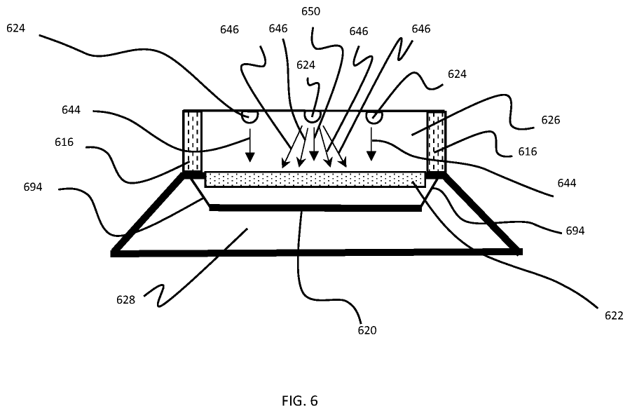

As shown in FIGS. 3A and 3B, in some embodiments, a filter frame 334 may be configured to releasably couple to the mask. The filter frame 334 shown in FIG. 3B may comprise at least an internal filter 322 (e.g., 122 in FIG. 1) secured in the filter frame 334. The filter frame may be configured to include fist filter 122 as shown in FIG. 1, internal filter 222 as shown in FIG. 2, filter 422 as shown in FIG. 4, filter 522 as shown in FIG. 5B, or filter 622 as shown in FIG. 6. The filter frame 334 may include at least one projection 338 that aligns with at least one slot 340 of the mask shown in FIG. 3A (e.g., via a slot in structural components 110 and/or 112 in FIG. 1, via a slot in mask frame 230 of FIG. 2, or via a slot in the perimeter of aperture 192, 292, 392, or 592 of FIGS. 1, 2, 3, and 5). The mask may be configured such that each of the at least one slots 340 can receive one of the at least one projections 338. The filter frame 334 may be rotated once received by slots 340, to secure the filter frame 334 to the mask. As shown in FIG. 4, in some embodiments, the filter frame 434 couples with the electronic module 414 such that a first filter 422 may be interposed between the electronic module 414 and the mask when the filter frame 434 is coupled to the mask. As described above, the filter frame and the electronic module are configured to include at least one projection while the mask includes at least one slot. In some embodiments, the mask may include the at least one projection while the filter frame and/or the electronic module include the at least one slot. The filter frame 434 may configured to be surrounded by the second filter 416. When the filter frame 434 is secured via the projections 438, the second filter 416 may also be secured.

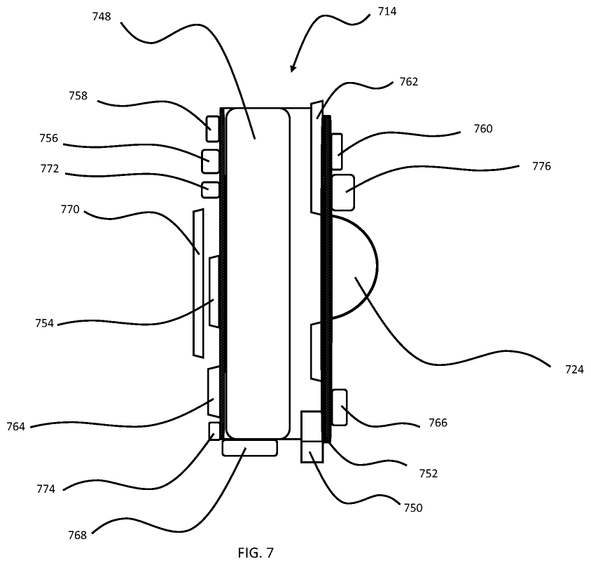

As shown in FIG. 3B, in some embodiments, a bio marker test strip 336 may be coupled to the internal filter 322. The bio marker test strip 336 may be embedded in the internal filter 322. The bio marker test strip 336 may be configured to detect where a user (e.g., 1090 in FIG. 10) has a virus or has been exposed to a virus. In some embodiments, the bio marker test strip 336 may be configured to detect COVID-19. In other embodiments, the bio marker test strip 336 may be configured to detect a user's blood sugar level. A spectral analytic sensor (e.g., 776 in FIG. 7) may analyze the bio marker test strip 336. For example, when an electronic module is coupled to a filter like internal filter 322 shown in FIG. 3A that comprises a bio marker test strip 336, the spectral analytic sensor may perform various forms of spectral analysis of test strip 336. A person of ordinary skill will appreciate that sensor may comprise both testing probes and meters, e.g., an illumination source and an optical sensor where the illumination source radiates the test strip at wavelengths that relate to the type of test strip and where the optical sensor detects the spectral response where the spectral response (as either absorption or emission) of the illumination source from the test strip changes based on the presence, absence, and/or concentration of a particular target substance. Other measurement techniques may also be integrated between the sensor and test strip 336 including electrical probes that measure changes in impedance across the test strip that relate to reactions with a particular target substance.

As shown in FIGS. 5A and 5B, in other embodiments, the mask may include a groove 542 located in aperture 592 (which corresponds to, for example, aperture 192 in FIG. 1 and aperture 292 in FIG. 2) that is configured to receive a filter frame 534 shown in FIG. 5A where the groove 542 defines a diameter smaller than the filter frame 534. The filter frame 534 may be configured to deform such that the filter frame 534 can be snapped in and out of the groove 542. As described above, the filter frame is configured to include a deformable frame while the mask includes a non-deformable groove. In some embodiments, the mask may include a deformable groove while the filter frame includes a set frame. Additionally, both the groove and the frame may be deformable. Alternatively, groove 542 may comprise a helical thread such that filter frame 534 may be screwed into aperture 592. In some embodiments, the material and/or geometry of filter frame 534 is configured to create an air tight seal with aperture 592 such that air passing through aperture 592 may only pass through filter 522. As described above, the groove is configured to include a helical thread while the filter frame is configured to mate with the helical thread. In some embodiments, the filter frame may include the helical thread while the groove of the mask is configured to receive the thread.

As shown in FIG. 6, the UV light source 624 may include one or more UV light sources 624. The UV light source 624 may be configured to produce one or more primary UV-C light beams 644 that are orthogonal to an internal filter 622. The UV light source 624 may also be configured to produce one or more secondary UV-C light beams 646 that interact with the internal filter 622 at an angle. The combination of the primary UV-C light beams 644 and the secondary UV-C light beams 646 operate to ensure maximum penetration of UV-C radiation into the internal filter 622 and/or maximum exposure to pathogens in chamber 626. The geometry and location of light source 624 may be adjusted from that shown to optimize exposure of UV-C lights on pathogens entering chamber 626. For example, UV-C light sources may also be disposed radially within the chamber 626 within the circumference of external filter 616. Similarly, in some embodiments UV-C light sources may be oriented away from a user (e.g., 1090 in FIG. 10), unlike the light source(s) 624 in FIG. 6 that are illustrated with the primary beams 644 oriented toward the user.

In some embodiments, the UV light source 624 includes at least one light emitting diode (“LED”). Radiation from the UV light source 624 may be configured to be contained within the chamber 626. The chamber 626 may include a reflective coating to increase UV-C radiation exposure within the chamber 626. In some embodiments, a reflective material may be positioned on sides of the internal filter 622 and the external filter 616 that face the chamber 626 to increase UV-C radiation exposure within the chamber 626. The internal filter 622 and the external filter 616 may be configured to contain the radiation from the UV light source 624. In some embodiments, a UV blocking plate 620 configured to block UV-C radiation may be interposed between the chamber 626 and the user (e.g., 1090 in FIG. 10). The UV blocking plate 620 may be made of a non-reflective, UV absorbing material or may include a UV absorbing coating. By containing the radiation from the UV light source 624, the user's exposure to UV radiation is limited. The UV blocking plate 620 may be supported by a UV blocking plate support 694. The UV blocking plate support 694 may support, define, and/or hold the UV blocking plate 620. The UV blocking plate support 694 may be coupled to the mask and may surround the entirety of the UV blocking plate 620. In some embodiments, the UV blocking plate support 694 may be coupled to the UV blocking plate 620 at a single or multiple locations around a perimeter of the UV blocking plate 620. The UV blocking plate support 694 may be configured to allow air to pass through. The UV blocking plate support 694 may, for example, be comprised of plastic, composite, metals, or a combination thereof. Further the UV blocking plate support 694 may comprise multiple subcomponents to provide the structure, frame, or support described by example herein.

As shown in FIG. 7, the electronic module 714 may be configured to power the UV light source 724. In some embodiments, the electronic module 714 may include the UV light source 724. The electronic module 714 may be configured to couple to an external battery where the external battery is configured to power the UV light source 724. In some embodiments, the electronic module 714 may include a battery 748. The battery 748 may be a rechargeable battery. In some embodiments the battery is integrated into the module while in other embodiments it may be external. Similarly, in some embodiments the battery is removable while in others it is not.

In an illustrative embodiment, the electronic module 714 for the air purification system may include a universal serial bus (“USB”) port 750. In some embodiments, the UV light source 724 may be configured to provide UV-C radiation to a chamber of a mask (e.g., 126 in FIG. 1). The UV light source 724 may include at least one light emitting diode (LED). The electronic module 714 may be configured to detachably couple to the mask. As shown in FIG. 7, in some embodiments, the electronic module 714 may include threads 752 configured to engage with the mask.

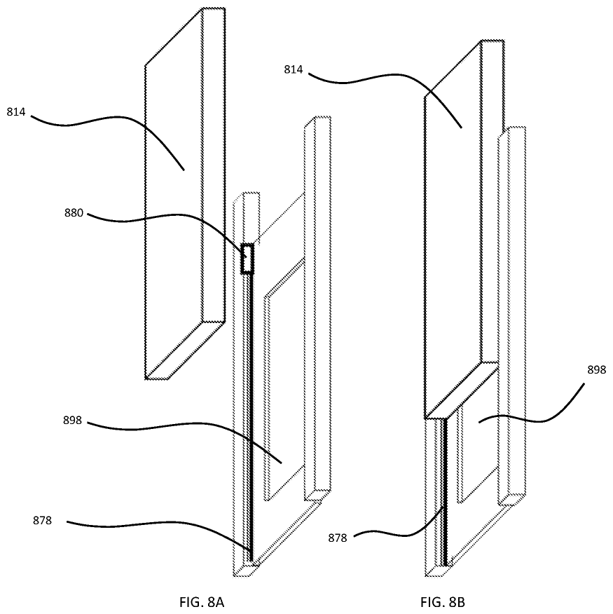

As shown in FIGS. 8A and 8B, in some embodiments, the electronic module 814 may be configured to couple to the mask by sliding into a track 878 of the mask. In some embodiments, the electronic module 814 may be configured to electrically connect to tools of the mask via an electrical coupling 898. The electronic module 814 in some embodiments includes a kill switch 880 configured such that when the electronic module 814 is fully seated in track 878, the UV-C light source (e.g., 724 in FIG. 7) may activate and when the electronic module 814 is disengaged from the mask, the UV-C light source may not activate. By making the electronic module 814 detachable, the user may remove the electronic module 814 for maintenance, electronic updates, and/or charging. Portions of the mask that are inaccessible when the electronic module 814 is coupled to the mask can be accessed when the electronic module 814 is removed. A person of ordinary skill will understand that the advantages and features of the implementation shown in FIGS. 8A and 8B are not limited to the depicted configuration and may be integrated into other implementations including those described and illustrated above with references to FIGS. 1, 2, 4, 6, and 7.

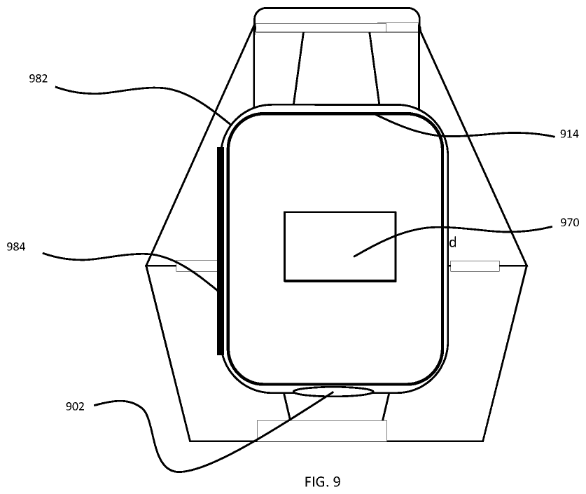

In some implementations, as shown in FIG. 9, the electronic module 914 may be configured to fit within a water resistant chamber 982. The water resistant chamber 982 enables the user to use the electronic module 914 in conditions where the mask may be exposed to water. For example, the user may continue using the electronic module 914 outdoors while it is raining. The water resistant chamber 982 may be configured to include hinge 984 for opening and closing the water resistant chamber 982. Latch 902 secures the water resistant chamber 982 and may be comprised of a push button, hook latch, slide latch, or the like. A person of ordinary skill in the art will appreciate that in some embodiments of the electronic modules described herein, such modules may be built with ingress protection, e.g., potting, conformal coating, lip-and-groove casings, waterproof gaskets, waterproof seals, and/or the like to water proof and/or dust proof the electronic module.

In various embodiments of the electronic modules disclosed herein, the battery (e.g., 748 in FIG. 7) may be configured to be charged via the USB port (e.g., 750 in FIG. 7). Additionally or alternatively, the battery may be configured to be wirelessly charged. Repeat use of the electronic module may be enabled by recharging the battery of the electronic module. In some implementations, the electronic module can be coupled to an external battery via the USB port. By enabling the electronic module to receive power via an external power source, the user can use continue using the electronic module when the user needs to use the electronic module for a duration longer than the life of the battery.

The electronic module may include a controller having a processor (e.g., a microcontroller/microprocessor, a central processing unit (CPU), a field-programmable gate array (FPGA) device, an application-specific integrated circuits (ASIC), another hardware device, a firmware device, or any combination thereof) and a memory (e.g., a computer-readable storage device) configured to store instructions, one or more thresholds, and one or more data sets, or the like. In some embodiments, the electronic module may include one or more interface(s), one or more I/O device(s), a power source, one or more sensor(s), or combination thereof. The electronic module may be physically or wirelessly coupled to one or more components of the electronic module and configured to control operation of the components via one or more user-initiated or automatic commands or parameters.

In some implementations, the components of the electronic modules described herein may include one or more sensors. For example, the electronic module (e.g., 714 in FIG. 7) comprises a camera (e.g. 756 in FIG. 7). As non-limiting examples, the camera may be an infrared camera, a laser based camera, a combination thereof, or a plurality of such cameras. In some embodiments, camera may be configured to record video within a field of view of the camera. By including the camera in a mask, the camera is better suited to capture a true first person perspective than traditional first person cameras that are mounted to a headband or a torso. In some embodiments, the camera may be configured to capture a thermal video within the field of view of the camera. The one or more sensors may be configured to face away from the user (e.g., 1090 in FIG. 10). For example, the camera may face away from the user. The outward facing sensor may provide information that enables the surrounding environment to be evaluated. Based on the surrounding environment, the mode of operation of the electronic module may be adjusted. In some implementations, by including one or more sensors, the electronic module may be configured to operate based on situational factors (e.g., triggers) detected by the one or more sensors.

In some embodiments, the one or more sensors may include a first sensor (e.g. 758 in FIG. 7) configured to measure a first air sample from the atmosphere and a second sensor (e.g., 760 in FIG. 7) configured to measure a second air sample from the user's respiration. The first sensor may be positioned on a portion of the mask exposed to an atmosphere and may be in direct fluid communication with the atmosphere. The second sensor may be positioned in an outer chamber (e.g., 126 in FIG. 1) between filters (e.g. 116 and 122 in FIG. 1) of some embodiments disclosed herein. By placing the first sensor in fluid communication with the atmosphere and the second sensor in fluid communication with the outer chamber, the electronic module is able to evaluate a difference between atmospheric conditions and conditions in the outer chamber of the mask. In some implementations, the electronic module may be able to determine what kind of particles are entering the outer chamber and how many particles are entering the outer chamber. In some implementations, the electronic module may be configured to determine, based on readings from the first sensor, the second sensor, or a combination thereof, whether the user is in a dangerous environment. For example, the electronic module may comprise a processor or interface with an external processing device that may compare measurements between the sensors against one another or against thresholds to determine whether hazardous components are in the environment and/or whether the hazardous components are being inhaled by the user. In other embodiments, based on readings from the first sensor, the second sensor, or a combination thereof, the electronic module may be configured to identify whether the user has a potential health risk. As a non-limiting example, the electronic module may be configured to determine whether a user's breathing corresponds to breathing of an individual with a disease. The electronic module may be configured to adjust a mode of operation based on the evaluation of the difference between the conditions outside the mask and the conditions in the in the outer chamber. The UV light source 724 may be configured to more efficiently eliminate, modify, disinfect, inactivate, or kill the unwanted particles by operating based on the evaluation of the difference between the conditions outside the mask and the conditions in the outer chamber.

The first sensor (e.g., 758 in FIG. 7) and the second sensor (e.g., 760 in FIG. 7) may include one of or any combination of the following: particle counter, temperature sensor, carbon dioxide sensor, airflow sensor, volatile organic compound sensor, ammonia sensor, carbon monoxide sensor, humidity sensor, oxygen sensor, combustible gas sensor, particle size sensor, smog sensor, atmospheric pressure sensor, radiation sensor, electromagnetic field sensor, or atmospheric pressure sensor. In some embodiments, the volatile organic compound sensor is configured to measure one of or any combination of the following: nitric oxide, nitrogen dioxide, pentene, trimethylamine, isoprene, isopropanol, acetaldehyde, benzene, methane, ethane, ethanol, hydrogen sulfide, or acetone.

In some embodiments, the electronic module may include a spectral analytic sensor (e.g. 776 in FIG. 7). The spectral analytic sensor may be configured to determine the results from a bio marker test strip (e.g., the bio marker test strip 336 shown in FIG. 3B). In some embodiments, the spectral characteristics of a bio marker test strip may be configured to change when a target substance is present and/or binds with the bio market test strip. In one embodiment, the bio marker test strip may be configured to change spectral characteristics based on the presence of a virus. The spectral analytic sensor may be configured to determine that the bio marker test strip has been exposed to a virus by detecting the change in spectral characteristics in the bio marker test strip. Other detection techniques, such as spectroscopy, electrical detection, assay, may be used alone or in combination using the features and sensors described herein.

In some implementations, the memory and the processor (e.g., 754 of FIG. 7) comprise a computing system and the camera (e.g., 756 in FIG. 7) may be coupled to the computing system. The computing system may be configured to initiate a recognition process based on a trigger event. Based on the initiation of the recognition process, the camera may be configured to capture an image. Based on the trigger event, the UV light source (e.g., 724 in FIG. 7) may be configured to activate or deactivate. By operating based on the trigger event and not all the time, the UV light source may last longer. For example, the life of the UV light source may be extended if the UV light source is only active if a person is within a certain distance. In some implementations, the camera may be configured to capture a video stream within a field of vision of the camera. The video stream may be configured to constantly run or run based on the trigger event. The above examples illustrate possible trigger events. Other trigger events are discussed below, and are provided as non-limiting examples

The recognition process may comprise a facial recognition process. The facial recognition process may include identifying an object that includes similarities to that of a geometric template. The geometric template may correspond to a face. In some implementations, based on the initiation of the recognition process, the computing system may be configured to receive the captured image and compare the captured image to at least one facial image. If a user (e.g., 1090 in FIG. 10) has or had a communicable disease, the facial recognition process can alert individuals whose faces were recognized and who came in contact with the user. In another embodiment, the electronic module may be configured to alert the user that the captured image corresponds to someone who has been identified as being at high risk of having a communicable disease. An individual may be identified as being at high risk based on information received by the electronic module via a wireless transceiver (e.g., 762 in FIG. 7) corresponding to a database of individuals who have either tested positive for a communicable disease or come in contact with someone who has tested positive for a communicable disease. In some embodiments, the mask may be configured to alert the user as to the specific communicable disease the high risk individual either has or has come in contact with.

The recognition process may comprise a temperature recognition process. When the recognition process is a temperature recognition process, the triggering event is a determination that a temperature of a captured image is greater than a threshold temperature. In some implementations, the threshold temperature is 100.3 degrees Fahrenheit. The threshold temperature may correspond with a body core temperature. The threshold temperature may be any temperature greater than an average body temperature (e.g., 98.6 degrees Fahrenheit). In addition to or alternative to the triggering event being a determination that a temperature of a captured image is greater than a threshold temperature, the triggering event may include a determination that the temperature of the captured image is less than a second threshold image. In some implementations, the second threshold temperature is 110 degrees Fahrenheit. The second threshold may be used to filter out noise, e.g., when trying to detect human temperatures above a certain threshold, impossible body temperatures may be ignored, such as heat detected from vehicle or exhaust unit via the second threshold temperature. In some implementations, the electronic module may be configured to provide an indication to the user when the temperature of the captured image is greater than a threshold temperature. The indication may include an audible, visible, or tangible notification. In other implementations, the electronic module 714 may be configured to provide the indication to the user when the temperature of the captured image is greater than a first threshold temperature and less than a second threshold temperature. By activating or deactivating the UV light source based on a temperature recognition process, the battery life may be extended by not depleting the battery when there is no benefit associated with the UV light source being active, such as when no one is around.

The recognition process may be a distance recognition process. When the recognition process is a distance recognition process, the triggering event is a determination that a distance between an object and the camera (e.g., 756 of FIG. 7) of the electronic module is within a threshold distance. In some implementations, the threshold distance is 6 feet. By activating or deactivating the UV light source based on a temperature recognition process, the battery life may be extended by not depleting the battery charge when there is no benefit associated with the UV light source being active, such as when no one is around.

The recognition process may be a location recognition process. When the recognition process is a location recognition process, the triggering event may be a determination that the user is indoors. In other embodiments, the electronic module may include a global positioning device configured to determine where a user is located. Based on user location, the electronic module may be configured to activate or deactivate By activating or deactivating the UV light source based on a temperature recognition process, the life of battery may be extended by not depleting the battery when there is no benefit associated with the UV light source being active, such as when no one is around.

The electronic module may be configured to operate as an internet of things (IOT) device. In some implementations, the electronic module may be configured to communicate with an external device. The external device may include one or more devices. The external device may include a removable flash drive (e.g., 764 of FIG. 7) where the electronic module is configured to receive the removable flash drive. In some implementations, the electronic module may be configured to communicate with the external device via a wireless transceiver (e.g., 762 of FIG. 7). The wireless transceiver may be configured to communicate with the external device via Bluetooth. In some implementations, the wireless transceiver may be configured to wirelessly communicate with an external network. The external network may include a cellular LTE network, a local area network, a cellular 5G network, a cloud network, or a combination thereof. In some implementations, the electronic module may be configured to receive data from the external device. As non-limiting examples, the data may include contract tracing data, data corresponding to a surrounding environment, weather data, pollution data, data corresponding to wanted individuals or persons of interest, or a combination thereof. By receiving data from the external device, the electronic module may be configured to alert a user of an existing hazard. In some implementations, the electronic module may be configured to receive tracing data providing that a particular individual has been exposed to a disease and alert the user if the potentially infected individual is close by. By receiving data from the external device, the electronic module may also be configured to alert the user of a future hazard. For example, the electronic module may be configured to alert the user of incoming inclement weather. In other implementations, the electronic module may be configured to alert the user of a dangerous individual in the area. In some implementations, the electronic module may be configured to activate or deactivate based on the data received from the external device.

The electronic module may include at least one micro-electromechanical system (“MEMS”) microphone (e.g., 766 in FIG. 7) disposed in an outer chamber (e.g., 126 in FIG. 1). The at least one MEMS microphone may be configured to capture a sound produced by the user (e.g., 1090 in FIG. 10) and reduce distortion of the sound produced by the user. Some embodiments of the present disclosure may use various combinations of pre or post filtering and/or pre or post amplification of sound detected by MEMS microphone. Further, such filtering and amplification may be performed digitally or through analog circuit design and may be performed locally in module or remotely on a connected processing device (e.g., a smartphone connected via Bluetooth). In some implementations, the electronic module may include an audio speaker (e.g., 768 in FIG. 7). The audio speaker may be positioned outside of the outer chamber (e.g., 126 in FIG. 1) and facing away from the user. In some implementations, the audio speaker may be configured to reproduce the captured sound produced by the user. By reproducing the sound produced by the user via an external speaker, a user wearing a mask can better communicate with others. In some implementations, the audio speaker is configured to produce an audible alarm based on a condition sensed by the electronic module. In an illustrative embodiment, the MEMS microphone may be configured to capture sounds whispered from an individual needing to remain quiet such as a soldier or a police officer. The sound captured via the MEMS microphone may be transmitted to team members of the whispering user (e.g., 1090 in FIG. 10).

In other embodiments, the electronic module may be configured to receive operational data corresponding to a particular team within a hospital. Based on the operational data received, the audio speaker may be configured to relay information identifying which medical staff are needed in a particular room. In another embodiment, the audio speaker may be configured to relay information identifying a type of medical procedure that is about to take place in a particular location.

The electronic module may be configured to transmit the captured sound produced by the user to the external device. In some implementations, the electronic module may be configured to control the external device based on the captured sound produced by the user. By controlling the external device via voice, the user may be able to eliminate the need to have a secondary device (e.g., a smart phone) with them at all times to control surrounding equipment. In some implementations, based on the captured sound produced by the user, the electronic module may be configured to activate or deactivate various components coupled to the electronic module.

The electronic module may include a display screen (e.g., 770 in FIG. 7 or 970 in FIG. 9). In some implementations, the display screen may be positioned on an exterior surface of the electronic module. The display screen may be configured to display a transcription of the captured sound produced by the user. By reproducing the sound produced by the user via an external screen, a user wearing a mask can better communicate with others. For example, the user may be able to communicate with others in a particularly loud environment where noise is hard to distinguish. The display screen may also enable the user to express opinions or relay funny messages. In other embodiments, the user may be able to push a message or a picture from a mobile device to the display screen to enable others to see the message or picture without the need to exchange germs as is typically the case when individuals trade mobile devices. In other embodiments, the display screen may be configured to display various colors, a team symbol, or a combination thereof.

In some implementations, the electronic module includes at least one light (e.g., 772 in FIG. 7) configured to face away from the user. By including the light in the module which is coupled to a mask, the light is better suited to capture a true first person perspective than traditional first person light that is mounted to a headband or a torso. The light may include at least one LED. In some implementations, the light may be configured to blink, change colors, or a combination thereof.

The electronic module may include a gesture sensor (e.g., 774 in FIG. 7). The gesture sensor may be configured to face away from the user. In some implementations, the gesture sensor is configured to capture a gesture performed by the user and the electronic module is configured to transmit an instruction to an external device based on the captured gesture.

As shown in FIG. 10, the electronic module 1014 may include a projector 1086 configured to face away from the user 1090. The projector 1086 may be configured to project beams of light 1006 onto a surface. The projected beams of light 1006 may form an image of graphical content 1088. The graphical content may be black and white or color. The graphical content 1088 may correspond to traveling directions. In some implementations, the graphical content 1088 may correspond to biological information. The graphical content 1088 may correspond with explosive location information. By displaying graphical content 1088, through the mask, the user 1090 may be able to receive instructions for performing certain tasks without a need to look at another device (e.g., smartphone) or a manual. In some implementations, the user may be able to display videos, pictures, and/or text from other devices coupled to the mask via the wireless transceiver. As a non-limiting example, the projector 1086 may be configured to project a keyboard onto a surface. The gesture sensor (e.g. 774 in FIG. 7) may be configured to capture the position of the user's 1090 fingers with respect to the projected keyboard. Projector 1086 may augment the field of view of the user 1090 with various graphical indications to guide a user or for the user to interact with. At least one benefit of projector 1086 and related augmented reality capabilities in the present disclosure is that a user who is already wearing a mask for other reasons, such as maintaining a sanitary environment, does not require additional hardware such as glasses or a handheld device.

1. An air purification system comprising:
a gasket configured to interface with a user's face;
a first filter;
a first chamber defined at least in part by the first filter and the gasket, the first chamber configured to be further defined in part by the user's face;
a second filter;
a second chamber defined at least in part by the first filter and the second filter;
an ultraviolet (“UV”) light source disposed in the second chamber; and
where the second filter, the second chamber, the first filter, and the first chamber are configured to direct airflow from atmosphere to a respiratory system of the user during inhalation.

2. The air purification system of claim 1, where the first filter is disposed between the first chamber and the second chamber.

3. The air purification system of claim 1, where the second filter is disposed between the second chamber and atmosphere.

4. The air purification system of claim 1, where the UV light source is capable of producing a primary UV-C light beam and the primary UV-C light beam is orthogonal to the first filter.

5. The air purification system of claim 1, where:
the first filter includes a first plurality of holes, the first plurality of holes configured to prevent a first size particle from passing through the first filter; and
the second filter includes a second plurality of holes, the second plurality of holes configured to prevent a second size particle from passing through the second filter, where the second size particle is larger than the first size particle.

6. The air purification system of claim 1, where the first filter is configured to prevent particles greater than 0.1 microns in size from passing through the first filter.

7. The air purification system of claim 1, where the second filter is configured to prevent particles greater than 5 microns in size from passing through the second filter.

8. The air purification system of claim 1, where the first filter and the second filter are configured to contain UV-C radiation from the UV light source within the second chamber.

9. The air purification system of claim 1, where the gasket is configured to form an air tight seal with the face of the user.

10. The air purification system of claim 1, where the gasket comprises a rubber.

11. The air purification system of claim 1, where the first filter comprises a membrane.

12. The air purification system of claim 1, where the second filter comprises a membrane.

13. The air purification system of claim 1, where UV light source comprises at least one light emitting diode (“LED”).

14. The air purification system of claim 1, where the first filter is configured to be removable.

15. The air purification system of claim 14, where the first chamber comprises a frame configured to hold the first filter.

16. The air purification system of claim 15, where the first filter is coupled to the frame and is further configured to release from the frame; the frame interposed between the first chamber and the second chamber and configured to be positioned on an opposite side of the first chamber than the user's face.

17. The air purification system of claim 15, where the frame comprises a slot configured to prevent movement of the first filter along two axes.

18. The air purification system of claim 17, further comprising a latch configured to secure the first filter to the first chamber when the latch is in a first position, and where the latch allows for removal of the first filter when in a second position.

19. The air purification system of claim 1, where the second filters are washable.

20. The air purification system of claim 1, further comprising an electronic module.

21. The air purification system of claim 20, where the electronic module is configured to power the UV light source.

22. The air purification system of claim 20, where the electronic module comprises the UV light source.

23. The air purification system of claim 20, where the electronic module is configured to be electronically coupled to an external battery.

24. The air purification system of claim 20, where the electronic module comprises a battery.

25. The air purification system of claim 24, where the battery comprises a rechargeable battery.

26. The air purification system of claim 1, where the first filter includes a bio marker test strip.

27. An electronic module for an air purification system, the electronic module comprising:
a battery;
an ultraviolet (“UV”) light source configured to provide UV-C radiation to a first chamber on a mask;
a USB port where:
the battery is configured to be charged via the USB port; and
the electronic module is configured to be detachably coupled to the mask.

28. The electronic module of claim 27, where the UV light source is a light emitting diode (“LED”).

29. The electronic module of claim 27, where the electronic module further comprises a water resistant enclosure.

30. The electronic module of claim 27, where the electronic module is configured to receive a removable flash drive.

31. The electronic module of claim 27, where the electronic module is configured to be coupled to an external battery via USB port.

32. The electronic module of claim 27, where the battery is configured to be wirelessly charged.

33. The electronic module of claim 27, further comprising a laser.

34. The electronic module of claim 27, further comprising an infrared sensor.

35. The electronic module of claim 27, further comprising a memory, a processor.

36. The electronic module of claim 35, further comprising a camera.

37. The electronic module of claim 36, where the camera is configured to face away from a user.

38. The electronic module of claim 36, where the camera is configured to capture a video stream within a field of vision of the camera.

39. The electronic module of claim 36, where:
the memory and the processor comprise a computing system;
the camera is coupled to the computing system;
the computing system is configured to initiate a recognition process based on a trigger event; and
based on an initiation of the recognition process, the camera is configured to capture an image.

40. The electronic module of claim 39, where the ultraviolet light source is configured to activate or deactivate based on the trigger event.

41. The electronic module of claim 39, where the recognition process comprises a facial recognition process.

42. The electronic module of claim 41, where the recognition process comprises identifying an object similar to a geometric template.

43. The electronic module of claim 41, where the computing system is configured to:
receive the captured image; and
compare the captured image to at least one facial image.

44. The electronic module of claim 39, where the recognition process comprises a temperature recognition process.

45. The electronic module of claim 44, where the trigger event comprises an identification that a temperature of a captured image is greater than a threshold temperature.

46. The electronic module of the claim 45, where the threshold temperature corresponds to body core temperature of 100.3 degrees Fahrenheit.

47. The electronic module of claim 45, where the electronic module is configured to provide an indication to a user when the temperature of a scanned image is greater than the threshold temperature.

48. The electronic module of claim 45, where the trigger event further comprises a second identification that the temperature of the captured image is less than a second threshold temperature.

49. The electronic module of claim 48, where the second threshold temperature corresponds to a body core temperature of 110 degrees Fahrenheit.

50. The electronic module of claim 48, where the electronic module is configured to provide an indication to a user when the temperature of a scanned image is greater than the threshold temperature and less than the second threshold temperature.

51. The electronic module of claim 39, where the recognition process comprises a distance recognition process.

52. The electronic module of claim 51, where:
the distance recognition process comprises determining a distance between an object in the image and the camera; and
the trigger event is that the distance between the object in the image and the camera is within a threshold distance.

53. The electronic module of claim 52, where the threshold distance comprises 6 feet.

54. The electronic module of claim 27, where the electronic module is configured to communicate with an external device.

55. The electronic module of claim 54 comprising a wireless transceiver.

56. The electronic module of claim 55, where the wireless transceiver is configured to communicate with the external device via bluetooth.

57. The electronic module of claim 56, further comprising:
at least one micro-electromechanical system (“MEMS”) microphone disposed in the first chamber, the at least one MEMS microphone configured to:
capture a sound produced by a user; and
reduce distortion of the sound cause by the first filter.

58. The electronic module of claim 57, where the electronic module is configured to transmit the captured sound produced by a user to the external device.

59. The electronic module of claim 57, where, based on the captured sound produced by a user, the electronic module is configured to activate or deactivate.

60. The electronic module of claim 57, where the electronic module is configured to control the external device based on the captured sound produced by a user.

61. The electronic module of claim 57, configured to interface with a display screen positioned on an exterior surface of the mask or on an exterior surface of the electronic module.

62. The electronic module of claim 61, where the display screen is configured to display a transcription of the sound produced by a user.

63. The electronic module of claim 57 further comprising an audio speaker.

64. The electronic module of claim 63, where the audio speaker is configured to reproduce the captured sound produced by a user.

65. The electronic module of claim 63, where the audio speaker is configured to produce an audible alarm based on a danger identified by the electronic module or the external device.

66. The electronic module of claim 56, where the electronic module is configured to receive data from the external device.

67. The electronic module of claim 66, where the data corresponds to contact tracing data.

68. The electronic module of claim 66, where the UV light source is configured to activate or deactivate based on the data from the external device.

69. The electronic module of claim 66, where the data corresponds to information about a surrounding environment.

70. The electronic module of claim 69, where the information based on the surrounding environment includes weather information.

71. The electronic module of claim 69, where the information based on the surrounding environment includes pollution information.

72. The electronic module of claim 56, further comprising a gesture sensor.

73. The electronic module of claim 72, where the gesture sensor is configured to face away from a user.

74. The electronic module of claim 72, where:
the gesture sensor is configured to capture a gesture performed by a user; and
the electronic module is configured to transmit an instruction to the external device based on the captured gesture.

75. The electronic module of claim 55, where the wireless transceiver is configured to wirelessly communicate with an external network.

76. The electronic module of claim 75, where the external network comprises a wireless local area network.

77. The electronic module of claim 75, where the external network comprises a cellular LTE network.

78. The electronic module of claim 75, where the external network comprises a cellular 5G network.

79. The electronic module of claim 27, further comprising a spectral analytic sensor.

80. The electronic module of claim 79, where the spectral analytic sensor is configured to evaluate a bio marker test strip.

81. The electronic module of claim 80, where the bio marker test strip is coupled to the mask.

82. The electronic module of claim 27, further comprising:
a plurality of sensors including at least a first sensor and a second sensor, where:
the first sensor is configured to measure a first air sample from atmosphere; and
the second sensor is configured to measure a second air sample from a mask user's respiration.

83. The electronic module of claim 82, where:
the first sensor is positioned on a portion of the mask outside of the first chamber; and
the first sensor is configured to be in direct fluid communication with the atmosphere.

84. The electronic module of claim 82, where:
the second sensor is disposed towards the first chamber.

85. The electronic module of claim 82, where:
the second sensor is disposed toward a second chamber defined at least in part by the first filter, a gasket of the mask configured to interface with a face of the user, and the face of the use.

86. The electronic module of claim 82, where the plurality of sensors includes at least a particle counter.

87. The electronic module of claim 82, where the plurality of sensors includes at least temperature sensor.

88. The electronic module of claim 82, where the plurality of sensors includes at least a carbon dioxide sensor.

89. The electronic module of claim 82, where the plurality of sensors includes at least an airflow sensor.

90. The electronic module of claim 82, where the plurality of sensors includes at least a volatile organic compound sensor configured to measure nitric oxide.

91. The electronic module of claim 82, where the plurality of sensors includes at least a volatile organic compound sensor configured to measure nitrogen dioxide.

92. The electronic module of claim 82, where the plurality of sensors includes at least a volatile organic compound sensor configured to measure pentene.

93. The electronic module of claim 82, where the plurality of sensors includes at least a volatile organic compound sensor configured to measure trimethylamine.

94. The electronic module of claim 82, where the plurality of sensors includes at least a volatile organic compound sensor configured to measure isoprene.

95. The electronic module of claim 82, where the plurality of sensors includes at least a volatile organic compound sensor configured to measure isopropanol.

96. The electronic module of claim 82, where the plurality of sensors includes at least a volatile organic compound sensor configured to measure acetaldehyde.

97. The electronic module of claim 82, where the plurality of sensors includes at least a volatile organic compound sensor configured to measure benzene.

98. The electronic module of claim 82, where the plurality of sensors includes at least a volatile organic compound sensor configured to measure methane.

99. The electronic module of claim 82, where the plurality of sensors includes at least a volatile organic compound sensor configured to measure ethane.

100. The electronic module of claim 82, where the plurality of sensors includes at least a volatile organic compound sensor configured to measure ethanol.

101. The electronic module of claim 82, where the plurality of sensors includes at least a volatile organic compound sensor configured to measure hydrogen sulfide.

102. The electronic module of claim 82, where the plurality of sensors includes at least a volatile organic compound sensor configured to measure acetone.

103. The electronic module of claim 82, where the plurality of sensors includes at least an ammonia gas sensor.

104. The electronic module of claim 82, where the plurality of sensors includes at least a carbon monoxide gas sensor.

105. The electronic module of claim 82, where the plurality of sensors includes at least a humidity sensor.

106. The electronic module of claim 82, where the plurality of sensors includes at least an oxygen gas sensor.

107. The electronic module of claim 82, where the plurality of sensors includes at least a combustible gases sensor.

108. The electronic module of claim 82, where the plurality of sensors includes at least a particle sensor configured to detect a particle size.

109. The electronic module of claim 82, where the plurality of sensors includes at least a smog sensor.

110. The electronic module of claim 82, where the plurality of sensors includes at least an atmospheric pressure sensor.

111. The electronic module of claim 82, where the plurality of sensors includes at least a radiation sensor.

112. The electronic module of claim 82, where the plurality of sensors includes at least an electromagnetic field (EMF) sensor.

113. The electronic module of claim 27, further comprising at least one LED configured to face away from a user.

114. The electronic module of claim 113, where the LED is configured to blink.

115. The electronic module of claim 113, where the LED is configured to change colors.

116. The electronic module of claim 27, further comprising a projector configured to face away from a user.

117. The electronic module of claim 116, where the projector is configured to display graphical content on a surface.

118. The electronic module of claim 117, where the graphical content corresponds to traveling directions.

119. The electronic module of claim 118, where the graphical content corresponds to biological information.

120. The electronic module of claim 117, where the graphical content corresponds a location of an explosive.

121. The electronic module of claim 82, where the plurality of sensors includes at least a carbon monoxide sensor, an atmospheric pressure sensor, an ammonia gas sensor, a carbon dioxide sensor, a temperature sensor, an airflow sensor, and a volatile organic compounds sensor.

122. The electronic module of claim 82, where:
the electronic module further comprises a wireless transceiver configured to communicate information measured by the first and second sensors to an external processor; and
the electronic module receives information for communicating a notification to a user where the information for communicating a notification is based, at least in part, on the results of the external processor processing the information measured by the first and second sensors.
